(19) United States
(12) Patent Application Publication
(10) Pub. No.: US 2004/0154946 A1
(43) Pub. Date: Aug. 12, 2004

(54) Storage apparatus for surgical implant device

(76) Inventors: Kenneth Solovay, Weston, FL (US); Gregory Madden, Ft. Lauderdale, FL (US); Thomas Jacobs, Delray Beach, FL (US); James Layer, Cooper City, FL (US); William Gregory, Ft. Lauderdale, FL (US)

Correspondence Address: Elizabeth Cary Miller, Esq., SUTHERLAND ASBILL & BRENNAN LLP, 999 Peachtree Street, NE, Atlanta, GA, 30309-3996, US

(21) Appl. No.: 10/702,982

(22) Filed: Nov. 6, 2003

Related U.S. Application Data
(60) Provisional application No. 60/424,283, filed on Nov. 6, 2002.

(51) Int. Cl. A61L 15/00

(52) U.S. Cl. 206/438

(57) ABSTRACT

The present invention is directed to a storage apparatus for a surgical implant device that protects the integrity of a surgical implant device during transport, provides protection of the surgical implant device prior to the use of the device in surgery, and facilitates the handling of the surgical implant device by the operator.

### CROSS-REFERENCE TO RELATED APPLICATIONS

This non-provisional application claims the benefit of U.S. Provisional Application No. 60/424,283 filed on Nov. 6, 2002.

### BACKGROUND OF THE INVENTION

1. Field of the Invention

The present invention is generally directed to a storage apparatus for a surgical device, and more specifically to an apparatus for storing a very small ophthalmic surgical implant device during shipment and prior to the use of the surgical implant device in surgery.

2. Background Art

Currently, there is no useful packaging in which to both safely transport a small surgical implant device and to facilitate handling of the surgical implant device during surgery. U.S. Pat. No. 6,221,078 to Bylsma et al. describes an apparatus for use in surgery to introduce a glaucoma drain into the eye. The apparatus comprises a tool with which to make a small wound in the eye. Following the creation of the wound, a glaucoma drain is loaded in the distal end of the apparatus and introduced into the wound by a plunger within the apparatus. Although this apparatus may be useful for delivery of a glaucoma drain, it would not be sufficient or practical for the safe transport, storage, or easy handling of the drain.

In PCT Publication No. US99/03592, Tormala et al. describe a packaging component for larger surgical implant devices such as pins, screws, tacks, nails, bolts, arrows, and plates. The packaging comprises a three dimensional mounting with perforations to removably receive the surgical implant. The packaging is useful for the transportation and storage of these surgical implant devices prior to and during surgery. Such a packaging would not be appropriate for smaller surgical implant devices such as a glaucoma shunt device.

What is needed in the art is a storage apparatus for a very small surgical implant device that protects the implant during shipping and which facilitates presentation and handling of the implant by medical staff during a procedure.

### SUMMARY OF THE INVENTION

The present invention is directed to a novel storage apparatus for a surgical implant device that protects the integrity of the surgical implant device during transport. The apparatus of the present invention also provides protection of the surgical implant device and maintains the implant device in a preferred configuration prior to the use of the device in surgery. The storage apparatus of the present invention further facilitates the presentation and handling of the surgical implant device by the operator.

### BRIEF DESCRIPTION OF THE DRAWINGS

FIG. 1A is an illustration showing the overhead perspective view of one embodiment of the present invention.

FIG. 1B is an illustration showing the overhead perspective view of one embodiment of the present invention engaged with a bi-directional glaucoma shunt device.

FIG. 1C is an illustration showing a cross sectional view of one embodiment of the present invention.

FIG. 2 is an illustration showing the overhead perspective view of the elongated barrel of one embodiment of the present invention.

FIG. 3 is an illustration showing the overhead perspective view of the elongated plunger of one embodiment of the present invention.

FIG. 4 is an illustration showing a close-up view of the retaining mounts of one embodiment of the present invention.

FIG. 5 is an illustration showing the overhead perspective view of one embodiment of the present invention, engaged with a bi-directional glaucoma shunt device.

FIG. 6 is an illustration showing the overhead perspective view of one embodiment of the present invention.

FIG. 7 is an illustration showing the overhead perspective view of the distal end of one embodiment of the present invention, engaged with a bi-directional glaucoma shunt device.

FIG. 8A is an illustration showing the overhead perspective view of one embodiment of the present invention, engaged with a bi-directional glaucoma shunt device.

FIG. 8B is an illustration showing a cross-sectional view of one embodiment of the present invention.

FIG. 9A is an illustration showing a cross-sectional view of one embodiment of the present invention, engaged with a bi-directional glaucoma shunt device.

FIG. 9B is an illustration showing a cross-sectional view of one embodiment of the present invention in the delivery position.

FIG. 10A is an illustration showing the overhead perspective view of one embodiment of the present invention.

FIG. 10B is an illustration showing a cross-sectional view of one embodiment of the present invention.

FIG. 11 is an illustration showing the overhead perspective view of one embodiment of the present invention.

FIG. 12 is an illustration showing the overhead perspective view of one embodiment of the present invention.

FIG. 13A is an illustration showing the overhead perspective view of one embodiment of the present invention in the closed position.

FIG. 13B is an illustration showing the overhead perspective view of one embodiment of the present invention in the open position.

FIG. 14A is an illustration showing the overhead perspective view of one embodiment of the present invention.

FIG. 14B is an illustration showing the overhead perspective view of one embodiment of the present invention.

FIG. 14C is an illustration showing the overhead perspective view of the main body portion of one embodiment of the present invention.

FIG. 15A is an illustration showing the overhead perspective view of one embodiment of the present invention.

FIG. 15B is an illustration showing the overhead perspective view of one embodiment of the present invention in the delivery position.

FIG. 16A is an illustration showing the overhead perspective view of one embodiment of the present invention.

FIG. 16B is an illustration showing the overhead perspective view of one embodiment of the present invention in the delivery position.

FIG. 17A is an illustration showing the overhead perspective view of one embodiment of the present invention.

FIG. 17B is an illustration showing the overhead perspective view of one embodiment of the present invention.

### DETAILED DESCRIPTION OF THE INVENTION

The present invention provides embodiments of a novel apparatus for storing a surgical implant device. In this embodiment, the storage apparatus comprises a base with a recessed surgical retaining mount and a cover which releasably engages the base. The device stored in this embodiment is a bi-direction shunt for use in diverting aqueous humor in the eye. The device of this embodiment uses two surgical implant retaining mounts. The surgical implant retaining mounts are continuous over the shape of the surgical implant device. The base of this embodiment has in addition to the surgical implant retaining mounts an access area adjacent to the recessed retaining mounts which aid in removal of the surgical implant device. The surgical implant device retaining mounts of this embodiment are, for example, at an angle of between about 10° and 150° from one another. In a more preferred form, the angel of the surgical implant device retaining mounts are at an angle of between about 30° and 120° from one another. Even more preferred, is the angle of the surgical implant device retaining mounts of between about 45° and 90° from one another. The surgical implant device retaining mounts of this embodiment are about 0.1 to 2.0 mm in diameter.

In another embodiment, the storage apparatus comprises an elongated barrel, an elongated plunger having recessed surgical implant device retaining mounts, and a nosepiece. The elongated barrel of one embodiment of the storage apparatus comprises a proximal end with a port, a narrower distal end with a port and a diameter transition portion therebetween. The elongated plunger of one embodiment of the storage apparatus consists of a proximal end, a distal end for movable insertion within the narrower distal port of the outer barrel, and a shaft therebetween for insertion within the proximal port of the barrel. The distal end of the plunger further comprises at least one recessed retaining mount for a surgical implant device. The nosepiece of the storage apparatus is configured to releasably engage the distal portion of the outer barrel, and functions as a protective cap. The releasable nosepiece, or cap, can releasably engage the outer barrel by frictionally retention or reciprocal screw threads, for example.

In another embodiment, the storage apparatus further comprises a biasing means. In a relaxed position, the biasing means maintains the distal end of the plunger within the distal end of the barrel, protecting a surgical implant device mounted in the retaining mounts on the distal end of the plunger. When force is applied to the biasing means, the plunger extends distally beyond the distal port of the outer barrel, exposing the surgical implant device in the retaining mounts. In still another embodiment, the biasing means is a coil spring attached at one end to the shaft of the plunger and at another end to the diameter transition portion of the barrel. The biasing means can be mounted in other positions on the apparatus and selected from any variety of such implements known in the art, such as springs elastic bands and the like.

In another embodiment of the present invention, the proximal portion of the barrel has an outer rim extending therefrom for gripping with an operator's fingers. In yet another embodiment, the proximal portion of the plunger has an outer rim extending therefrom for pushing with an operator's thumb. In another embodiment of the present invention, the shaft of the plunger is prevented from moving within the narrower distal end of the barrel by abutting the diameter transition portion.

In another embodiment of the present invention, the elongated barrel comprises polycarbonate material. The proximal end and shaft of the elongated plunger of this embodiment comprise polycarbonate material, and the distal end comprises silicone rubber. The nosepiece of this embodiment comprises polyethylene and may further comprise an elastimer additive.

In an alternative embodiment, the invention provides an apparatus for storing a surgical implant device, wherein the apparatus comprises a base comprising at least one recessed surgical device retaining mount and a cover configured to releasably engage the base. In this embodiment, there are no reciprocating plunger and barrel features, and the implant device is stored and presented on the base, while the cover protects the implant device during storage.

In certain embodiments of the present invention, the storage apparatus contains at least one surgical implant device retaining mount recessed into the base or distal end of the plunger. In another embodiment, the storage apparatus comprises two surgical implant device retaining mounts recessed into the base or distal end of the plunger at an angle of between about 10° and 150° from one another. More preferably, the angle between the surgical implant device retaining mounts is between about 30° and 120° from one another. Even more preferably, the angle between the surgical implant device retaining mounts is between about 45° and 90° from one another. In one embodiment, the angle between the surgical implant device retaining mounts is about 800 from one another. The angled retaining mounts assist the surgical implant device in maintaining a preferred configuration for surgical implantation.

In yet another embodiment of the present invention, a surgical implant device is mounted in the distal end of the elongated plunger. The surgical implant device is fully enclosed within the barrel and nosepiece when in a storage position and may be at least partially exposed through the distal port of the barrel by removing the nosepiece and moving the plunger distally.

The present invention can be used in conjunction with a surgical implant device, such as a bi-directional shunt device described in U.S. Pat. No. 6,450,984, incorporated by reference herein in its entirety, used to divert aqueous humor in the eye from the anterior chamber into Schlemm's canal. Such a device comprises a distal portion having at least one terminal aspect sized and shaped to be circumferentially received within a portion of Schlemm's canal, and a proximal portion having at least one terminal aspect sized and shaped to be received within the anterior chamber of the eye, wherein the device permits fluid communication between the proximal portion in the anterior chamber to the distal portion in Schlemm's canal. Such a device, for example, can have a distal portion of between about 1 and 40 mm in length. The diameter of the distal portion, for example, can be between about 0.1 mm to 0.5 mm.

During transport and storage, the distal portion of this shunt device would be held stationary by the retaining mounts of the present invention. The shunt device is fully enclosed within the barrel and nosepiece when in a storage position. Therefore, the retaining mounts of the apparatus of the present invention are preferably sized and shaped so as to accommodate the ophthalmic implant device described in U.S. Pat. No. 6,450,984. For example, the retaining mounts of the apparatus of the present invention can be between about 1 mm to 40 mm in depth, more preferably between about 2 mm to 20 mm in depth. The retaining mounts of the apparatus of the present invention can be between about 0.1 to 2.0 mm in diameter, more preferably between about 0.2 mm and 1.0 mm in diameter, and more preferably between about 0.3 mm and 0.5 mm in diameter. In one embodiment, the retaining mounts of the apparatus are about 0.38 mm in diameter.

During surgery, the shunt device is at least partially exposed by removing the nosepiece, enabling the safe and efficient handling by the operator. In certain embodiments, the shunt device is at least partially exposed through the distal port of the barrel by removing the nosepiece and moving the plunger distally, enabling the safe and efficient handling by the operator.

In another embodiment, the invention provides an apparatus for storing a surgical implant device, wherein the apparatus comprises at least one mandrel configured to releasably engage a pore of a surgical implant device, such as the fluid communication channel of the distal portion(s) of a shunt. In one embodiment, the apparatus further comprises a means to remove the surgical device implant from the mandrel, and release the surgical device onto the eye, such as a telescoping sleeve around the outside of the mandrel. The diameter of the mandrel is approximately the diameter of the pore of the surgical implant device, or alternatively, the mandrel is tapered, so that the mandrel inserts into the pore of the surgical implant device to a position where the device is held firmly by the mandrel.

In another embodiment, the invention provides for an apparatus for storing a surgical implant device, wherein the apparatus comprises an elongated substantially tubular member having distal and proximal ends. Preferably each end of the tubular member contains a port, wherein the tubular member further comprises a lumen extending between the proximal end and distal end. The embodiment preferably further comprises an elongated plunger having a proximal end, a distal end for movable insertion within the distal port of the tubular member, and a shaft therebetween for insertion of the plunger within the proximal port of the tubular member. The apparatus provides that a surgical implant device can be located within the lumen at the distal end of the tubular member. In a preferred embodiment, the surgical implant device is fully enclosed within the lumen of the tubular member when in a storage position, and the surgical implant device is at least partially exposed through the distal port of the elongated substantially tubular member by moving the plunger distally when in a delivery position.

Another embodiment of the present invention provides an apparatus for storing a surgical implant device, wherein the apparatus comprises an elongated substantially tubular member having distal and proximal ends. Preferably each end of the tubular member contains a port, wherein the tubular member further comprises a lumen extending between the proximal end and distal end. The embodiment preferably further comprises a plurality of articulated members comprising a grasping element located at the distal end of the tubular member, wherein the grasping element comprises a tension means for holding a surgical implant device within the tubular member. In a preferred embodiment, the plurality of articulated members are connected to the distal end of a shaft wherein the shaft extends to the proximal end of the tubular member. In a further preferred embodiment, the invention comprises a manual actuation means located at the proximal end of the shaft for releasing the tension means of the grasping element. In a preferred embodiment, the surgical implant device is fully enclosed within the lumen of the elongated substantially tubular member when in a storage position, and the surgical implant device is at least partially exposed through the distal port of the elongated substantially tubular member by moving the shaft distally by using the manual actuation means to release the tension means of the grasping element when in a delivery position. In a further preferred embodiment, the manual actuation means allows removal of the surgical implant.

Another embodiment of the present invention provides an apparatus for storing a surgical implant device, wherein the apparatus comprises a substantially planar mounting surface comprising a plurality of a securing pins for supporting a surgical implant device, wherein the surface is releasably enclosed within a carriage. In a preferred embodiment, the surface of the apparatus comprises three securing pins. In a further preferred embodiment, a securing pin comprises a peg hook. Preferably, a carriage comprises a substantially planar covering surface, a plurality of side surfaces, and a pair of engagement members for slidably engaging the substantially planar covering across the planar counting surface to protect the surgical implant. In one embodiment, one of the side surfaces further comprises a stop member. In a further embodiment, the apparatus further comprises a handle attached to the carriage.

Another embodiment of the present invention provides an apparatus for storing a surgical implant device, wherein the apparatus comprises an elongated substantially tubular member having distal and proximal ends, each end containing a port, wherein the elongated tubular member further comprises a lumen extending between the proximal end and distal end. Preferably, the apparatus further comprises an elongate main body portion having distal and proximal ends, the distal end for movable insertion within the distal port of the tubular member. In a preferred embodiment, the apparatus further comprises a unitary substantially clam shell-shaped engagement device adapted to releasably engage the surgical implant device and including a pair of ends defining an opening therebetween, a mid-portion secured to the distal end of the elongate main body portion and an interior region defining a size corresponding to at least the width of the surgical implant device, wherein the mid-portion is secured to the elongate main body portion by a hinge portion. In a preferred embodiment of the invention, the surgical implant device is fully enclosed within the interior portion of the clam shell-shaped engagement device, which is in turn enclosed within the lumen of the elongated substantially tubular member when in a storage position. Preferably, moving the elongate main body portion distally at least partially exposes the clam shell-shaped engagement device, and releases the hinge portion to at least partially expose the surgical implant device when in a delivery position.

Another embodiment of the present invention provides an apparatus for storing a surgical implant device, wherein the apparatus comprises an elongated substantially tubular member having distal and proximal ends, each end containing a port, wherein the elongated tubular member further comprises a lumen extending between the proximal end and distal end. The apparatus preferably further comprises an elongate main body portion having distal and proximal ends, the distal end for movable insertion within the distal port of the tubular member; and an engagement device adapted to releasably engage the surgical implant device wherein the engagement device comprises a plurality of jaws, where each jaw comprises a distal end, a proximal end secured to the distal end of the elongate main body portion by a hinge portion, and wherein the plurality of jaws circumscribe an interior region defining a size corresponding to at least the width of the surgical implant device. In a preferred embodiment, the surgical implant device is fully enclosed within the interior portion of the engagement device, which is in turn enclosed within the lumen of the elongated substantially tubular member when in a storage position. In a further preferred embodiment, the engagement device is at least partially exposed through the distal port of the elongated substantially tubular member and releases the hinge portion to at least partially expose the surgical implant device by moving the elongate main body portion distally when in a delivery position.

Another embodiment of the present invention provides an apparatus for storing a surgical implant device, wherein the apparatus comprises an elongated substantially tubular member having distal and proximal ends, each end containing a port, wherein the elongated tubular member further comprises a lumen extending between the proximal end and distal end. The apparatus preferably further comprises an elongated main body portion having distal and proximal ends, the distal end for movable insertion within the distal port of the tubular member; and an articulable container adapted to releasably engage the surgical implant device, wherein the articulable container comprises a multi-plane surface and a transverse surface. In a preferred embodiment, the multi-plane surface comprises an upper surface, a plurality of hinge lines, and a plurality of lower surfaces. In a further preferred embodiment, the multi-plane surface and the transverse surface surround an interior region defining a size corresponding to at least the width of the surgical implant device, and the transverse surface is attached to the distal end of the elongate main body portion. In a preferred embodiment, the surgical implant device is fully enclosed within the interior region of the articulable container, which is in turn enclosed within the lumen of the elongated substantially tubular member when in a storage position. In a further preferred embodiment, the articulable container is at least partially exposed through the distal port of the elongated substantially tubular member, and releases the plurality of hinge lines to at least partially expose the surgical implant device by moving the elongate main body portion distally when in a delivery position.

Another embodiment of the present invention provides an apparatus for storing a surgical implant device, wherein the apparatus comprises a substantially planar stage portion, wherein the stage portion has an external key opening; and an elongated substantially tubular member at least partially located within the stage portion, wherein the tubular member rotates within the stage portion of the apparatus, and wherein the tubular member has an internal key opening located in the midportion of the tubular member. In a preferred embodiment, the tubular member has an interior region comprising an engagement means for holding the surgical implant device, wherein the engagement means comprises a surface adapted to releasably engage the surgical implant device; and the apparatus comprises an positioning means for aligning the internal key opening of the tubular member with the external key opening of the stage portion when the tubular member is rotated within the stage portion. In a preferred embodiment, upon the alignment of the positioning means, the surgical implant device is exposed through the internal key opening and the external key opening of the apparatus.

Another embodiment of the present invention provides an apparatus for storing a surgical implant device, wherein the apparatus comprises a top portion comprising an outer surface and an inner surface, the inner surface adapted to releasably engage the surgical implant device, and the outer surface having a convex portion for pushing with an operator's thumb. In a preferred embodiment, the apparatus further comprises a bottom portion comprising an outer surface and a substantially horizontal inner surface. Preferably, an interior region of the apparatus is defined by the inner surface of the top portion and the inner surface of the bottom portion. In a preferred embodiment, the apparatus further comprises an engagement means, wherein the engagement means comprises one or more receiving portions and one or more corresponding engagement members for slideably engaging the receiving portions. The one or more receiving portions can be located on the top portion and the one or more corresponding engagement members can be located on the bottom portion or alternatively, the one or more receiving portions can be located on the bottom portion and the one or more corresponding engagement members can be located on the top portion. In a preferred embodiment, upon the sliding of the engagement means, the surgical implant device is exposed through the inner surface of the top portion of the apparatus when in a delivery position.

Another embodiment of the present invention provides an apparatus for storing a surgical implant device, wherein the apparatus comprises an elongated substantially tubular member having distal and proximal ends, the proximal end containing a port, wherein the elongated tubular member further comprises a lumen extending between the proximal end and distal end, and wherein the tubular member has an external key opening located distally. Preferably, the apparatus further comprises an elongated main body portion having distal and proximal ends, the distal end for movable insertion within the proximal port of the tubular member, and wherein the distal end comprises a engagement means for holding the surgical implant device comprising a substantially horizontal surface adapted to releasably engage the surgical implant device. In a further embodiment, at least one end of the tubular member and the corresponding end of the elongate main body portion includes a positioning means for aligning the tubular member with the elongate main body portion when the elongate main body portion is rotated within the tubular member. Preferably, when the positioning means is aligned, the main body portion is moved distally to expose the surgical implant device through the external key opening of the tubular member.

Another embodiment of the present invention provides an apparatus for storing a surgical implant device, wherein the apparatus comprises a top portion comprising a substantially planar surface, having an external key opening, and comprises a bottom portion comprising a substantially planar surface, wherein the planar surface of the bottom portion has a interior region comprising an engagement means for holding the surgical implant device. Preferably, the engagement means comprises a surface adapted to releasably engage the surgical implant device. In a further preferred embodiment, the apparatus comprises a rotatable engagement means for attaching the top portion to the bottom portion, wherein the engagement means comprises a receiving portion and an engagement member for rotatably engaging the receiving portion, wherein rotating the engagement means exposes the surgical implant device contained within the interior region through the external key opening of the top portion of the apparatus. In a preferred embodiment, the top portion and the bottom portion further comprise a side portion, wherein the side portions are located on opposite sides of the apparatus, and function as an alignment means for the apparatus.

Another embodiment of the present invention provides an apparatus for storing a surgical implant device, wherein the apparatus comprises a bottom portion comprising a substantially circular planar surface having an external key opening; a top portion comprising a parallel substantially circular planar surface; and a middle portion comprising a parallel substantially circular planar surface, wherein at least part of the middle portion extends beyond the top portion and the bottom portion, and wherein the middle portion has one or more engagement means for holding the surgical implant device, wherein the engagement means comprises an interior region adapted to releasably engage the surgical implant device. In a preferred embodiment, the apparatus further comprises a rotatable engagement means for attaching the top portion, bottom portion, and middle portion, wherein rotating the engagement means exposes the surgical implant device contained within the interior region of the middle portion through the external key opening of the bottom portion of the apparatus for removal of the surgical implant device.

Another embodiment of the present invention provides an apparatus for storing a surgical implant device, wherein the apparatus comprises a base and a cover releasably attached to the base. The base has as a retaining mount a recessed portion shaped similarly to the surgical implant device being stored therein. The size of this recessed portion is such that the surgical implant device fits snugly yet allows the surgical implant device to be removed without damaging the device. This recessed portion, or securing means, may be in the form of securing mounts, clips, latches, ties, binders, pinchers, fasteners, or any other means to releaseably attach and detach a surgical implant device. The securing mounts, for example, may be branched with an angle between the elements of the surgical implant device retaining mounts of between about 10° and 150° from one another. More preferably, the angle between the surgical implant device retaining mounts is between about 30° and 120° from one another. Even more preferably, the angle between the surgical implant device retaining mounts is between about 45° and 90° from one another. In one embodiment, the angle between the surgical implant device retaining mounts is about 80° from one another. The further recessed portion of the base contains at least one additional access area which increases the ease of removal of the surgical implant device from the base. The size of the access area which aids in removal of the surgical implant device can vary depending on how the device needs to be handled once removed. If the device is to be handled with forceps or other surgical pick-up devices, then the access area to aid in that removal may be smaller than if the surgical implant device is to be handled manually. The base portion can be equipped with fastening slots to engage reciprocating tabs extending from the cover to close or lock down the cover.

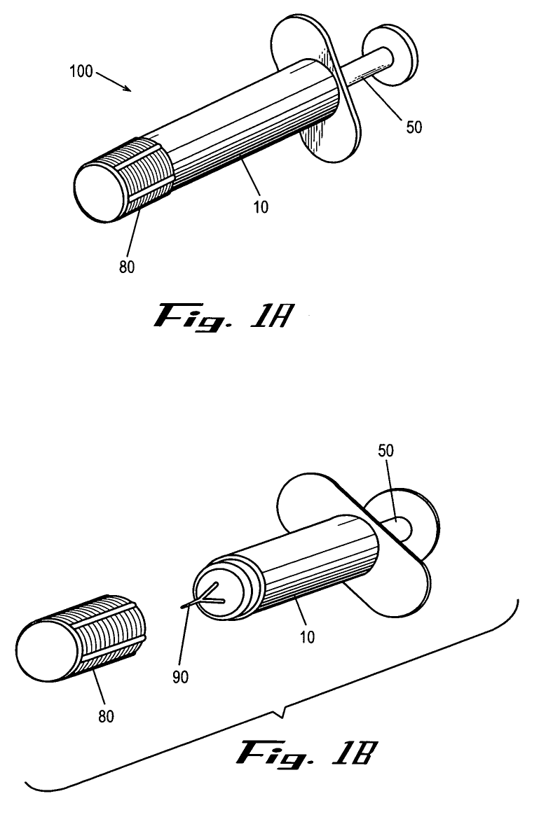

One embodiment of the present invention is illustrated in FIG. 1A, in which the storage apparatus 100 is shown in an overhead perspective view. The storage apparatus 100 of this embodiment comprises an elongated barrel 10, an elongated plunger 50, and a nosepiece 80. FIG. 1B illustrates this embodiment in which the storage apparatus 100 is shown in an overhead perspective view, engaged with a shunt device 90 used in the treatment of glaucoma. FIG. 1C illustrates this embodiment in which the storage apparatus 100 is shown in a cross sectional view. The storage apparatus 100 of this embodiment further comprises a coil spring 85 that is attached at one end to the shaft 65 of the plunger 50 and at the other end to the diameter transition portion 30 of the elongated barrel 10.

The elongated barrel 10 of one embodiment of the present invention is illustrated in FIG. 2, in which the elongated barrel 10 is shown in an overhead perspective view. The elongated barrel 10 further comprises a proximal end 15, a distal end 40, and a diameter transition portion 30 therebetween. The proximal end 15 of the elongated barrel 10 of this embodiment comprises a port 20 and an outer rim 25. The outer rim 25 of the barrel 10 of this embodiment extends from the barrel 10 and is of appropriate size to be gripped by the operator's fingers.

The elongated plunger 50 of one embodiment of the present invention is illustrated in FIG. 3, in which the elongated plunger 50 is shown in an overhead perspective view. The elongated plunger 50 further comprises a proximal end 55, a distal end 75, and a shaft 65 therebetween. The proximal end 55 of the elongated plunger 50 of this embodiment comprises an outer rim 60 that extends from the plunger 50 and is of appropriate size to be pushed by the operator's thumb. The distal end 75 of the elongated plunger 50 of this embodiment comprises at least one retaining mount 70.

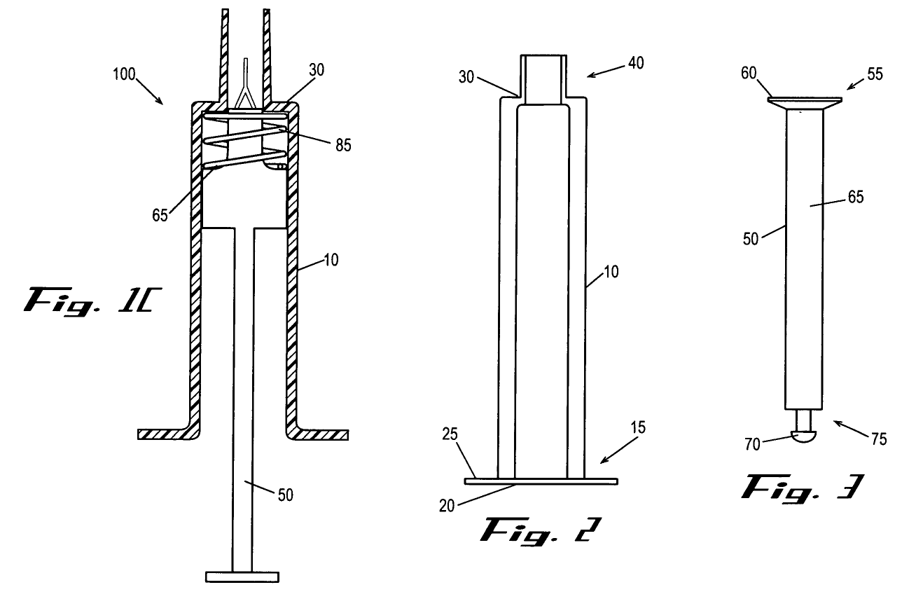

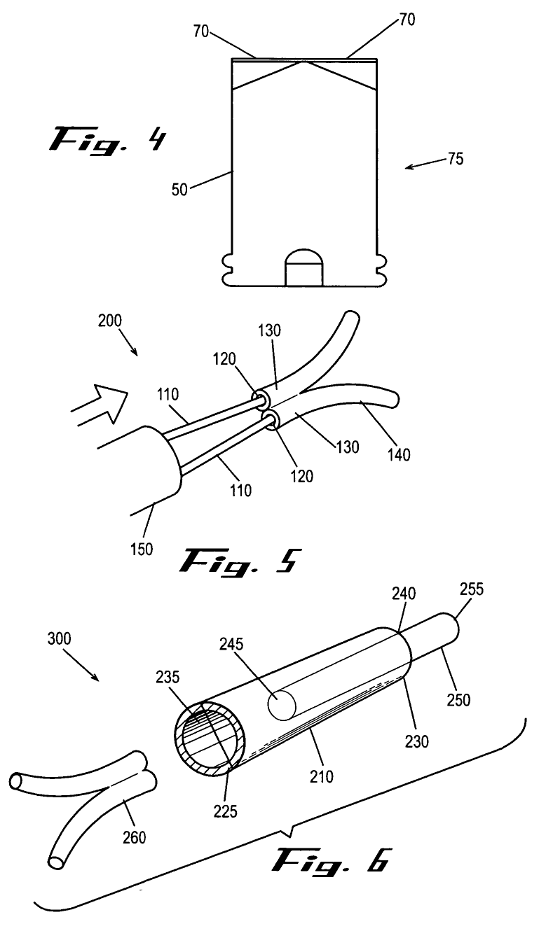

FIG. 4 illustrates a close-up view of the distal end 75 of the elongated plunger 50 of one embodiment of the present invention which comprises two retaining mounts 70. The retaining mounts 70 of this embodiment of the storage apparatus 100 are recessed into the distal end 75 of the plunger 50 at an angle of approximately 800 from one another.

FIG. 5 illustrates an overhead perspective view of one embodiment of the invention. The storage apparatus 200 comprises at least one mandrel 110. The mandrel engages the fluid communication channel 120 of the distal portion(s) 130 of a shunt device 140 used to treat glaucoma. A telescoping sleeve 150 around the outside of the mandrel is shown as an example of a means to remove the shunt device 140 from the mandrel 110 and release the shunt device onto the eye.

FIG. 6 illustrates another embodiment of the invention in an overhead view. The storage apparatus 300 comprises an elongated substantially tubular member 210 having a distal 225 and proximal end 230, each end containing a port. The distal port 235 and proximal port 240 of the tubular member are shown. The distal end 245 of a plunger 250 is inserted within the proximal port 240 of the tubular member 210. The proximal end 255 of the plunger 250 extends from the proximal port 240 of the tubular member 210. The distal port 235 of the tubular member 210 is engaged with a shunt device 260 used in the treatment of glaucoma.

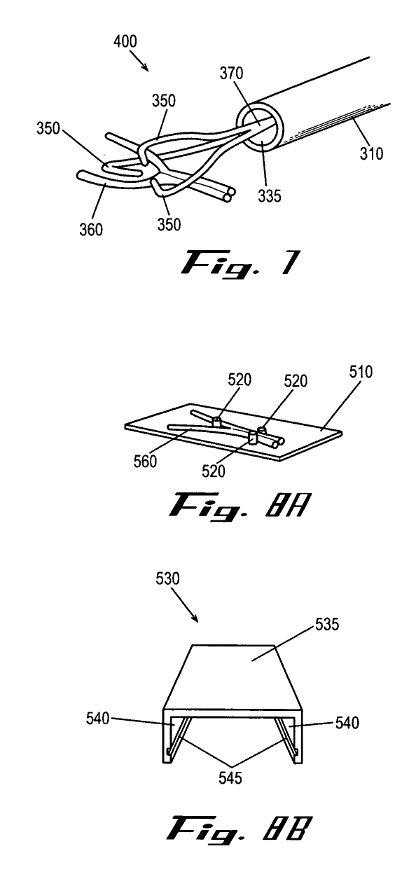

The storage apparatus 400 of another embodiment of the present invention is illustrated in FIG. 7, in which the tubular member 310 is shown in an overhead view. The distal end 335 of the tubular member 310 is shown. The apparatus further comprises a plurality of articulated members 350 located at the distal end 335 of the tubular member 310. A shunt device 360 used in the treatment of glaucoma is shown engaged within the articulated members. The articulated members 350 are connected to the distal end of a shaft 370 extending into the distal end 335 of the tubular member 310.

FIG. 8A illustrates an overhead view of another embodiment storage apparatus 500 of the present invention. The apparatus 500 comprises a substantially planar mounting surface 510 comprising a plurality of a securing pins 520 for supporting a shunt device 560 used in the treatment of glaucoma. FIG. 8B illustrates a cross-sectional view of the carriage 530 for enclosing the planar mounting surface shown in FIG. 8A. The carriage comprises a substantially planar covering surface 535, a plurality of side surfaces 540, and a pair of engagement members 545.

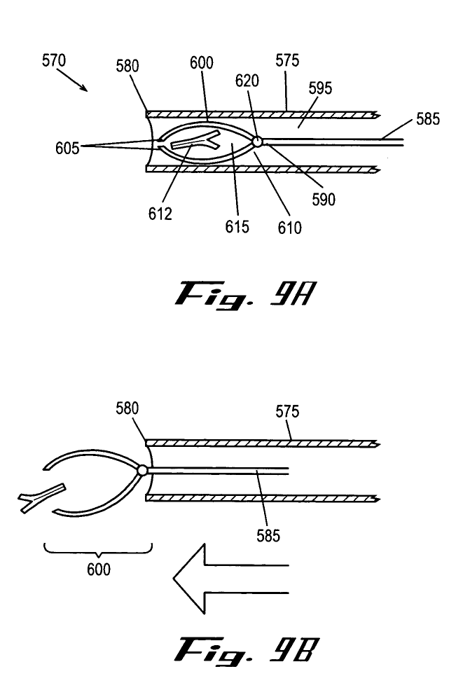

FIG. 9A illustrates a longitudinal view of another storage apparatus embodiment. The apparatus 570 comprises an elongated substantially tubular member 575. The distal end 580 of the tubular member 575 is shown. The elongate main body portion 585 having a distal end 590, is shown within the lumen 595 of the tubular member 575. The apparatus further comprises a unitary substantially clam shell-shaped engagement device 600, which includes a pair of ends 605, a mid-portion 610 secured to the distal end 590 of the elongate main body portion, and an interior region 615. The mid-portion 610 is secured to the elongate main body portion 585 by a hinge portion 620. A shunt device 612 used in the treatment of glaucoma is shown engaged within the interior region 615 of the clam shell-shaped engagement device 600. FIG. 9B illustrates a longitudinal view of the apparatus. The elongate main body portion 585 has moved distally, and the clam shell-shaped engagement device 600 is at least partially exposed past the distal end 580 of the tubular member 575.

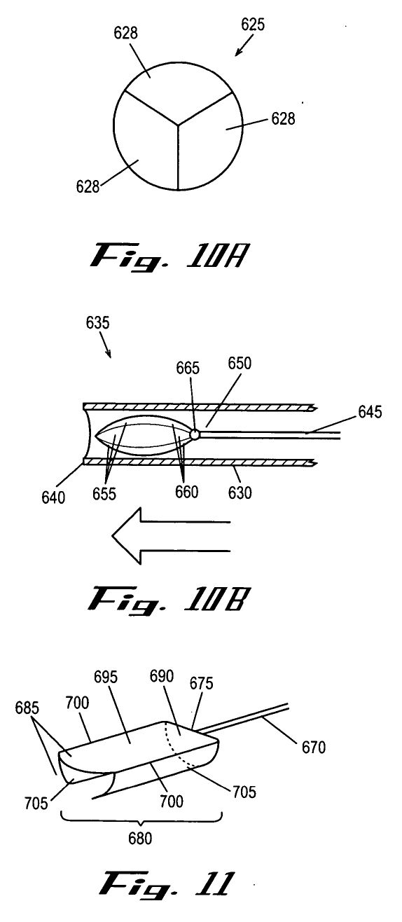

The engagement device 625 of another embodiment is shown in an overhead view in FIG. 10A. The engagement device comprises a plurality of jaws 628. FIG. 10B illustrates an longitudinal view of the storage apparatus 635 comprising the engagement device. The storage apparatus 635 comprises a tubular member 630. The distal end 640 of the tubular member 630 is shown. An elongate main body portion 645 having a distal end 650 is located within the tubular member 630. The engagement device is attached to the distal end 650 of the main body portion 645 by a hinge portion 665. Each jaw 630 of the engagement device comprises a distal end 655, and a proximal end 660.

FIG. 11 illustrates an overhead view of another storage apparatus. The apparatus comprises a main body portion 670, attached at its distal end 675 to an articulable container 680. The articulable container 680 comprises a multi-plane surface 685, and a transverse surface 690. The multi-plane surface 685 comprises an upper surface 695, a plurality of hinge lines 700, and a plurality of lower surfaces 705.

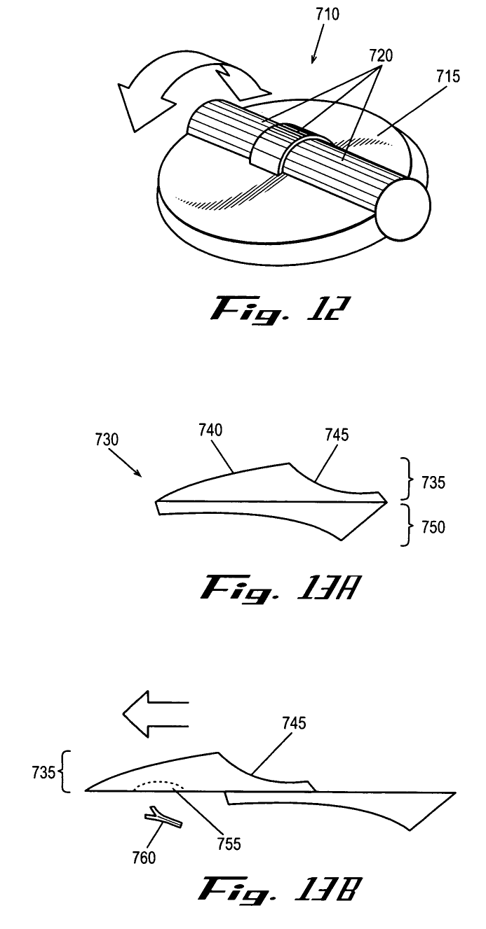

FIG. 12 illustrates an overhead view of another storage apparatus 710. The storage apparatus comprises a substantially planar stage portion 715; and an elongated substantially tubular member 720 at least partially located within the stage portion. The tubular member 720 rotates within the stage portion 715 of the apparatus.

FIG. 13A illustrates an overhead view of another storage apparatus 730 in the closed position. The apparatus comprises a top portion 735, where the outer surface 740 is shown. The outer surface 740 has a convex portion 745 for pushing with an operator's thumb. The apparatus further comprises a bottom portion 750, where the outer surface is shown. FIG. 13B illustrates an overhead view of the storage apparatus in the open position. By pushing on the convex portion 745, the apparatus opens, exposing the inner surface 755 of the top portion 735 and the shunt device 760 used in the treatment of glaucoma stored within the storage apparatus.

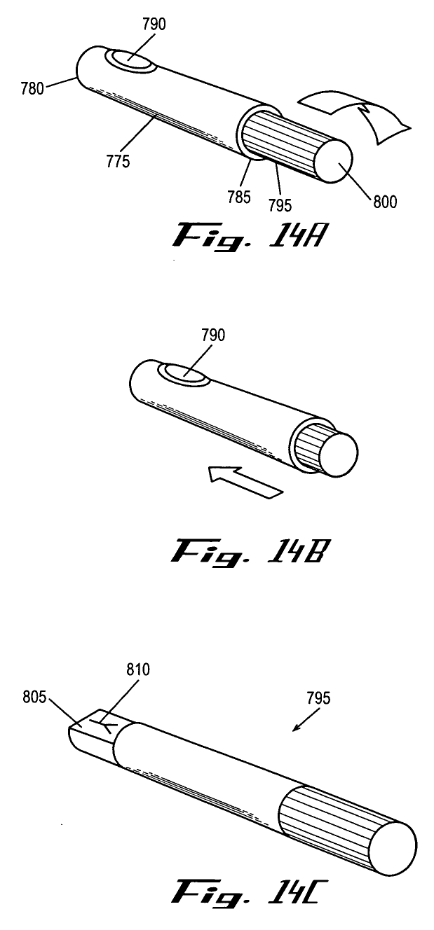

FIG. 14A illustrates an overhead view of a storage apparatus 770. The storage apparatus comprises a tubular member 775 having distal 780 and proximal 785 ends. The tubular member 775 has an external key opening 790 located distally. The apparatus further comprises a main body portion 795, with its proximal end 800 indicated. The proximal end 800 rotates and inserts within the proximal end 785 of the tubular member. FIG. 14B illustrates an overhead view of the apparatus, showing that after the main body portion is rotated within the tubular member and aligned, the main body portion is inserted distally within the tubular member to expose the shunt device through the external key opening 790. FIG. 14C illustrates an overhead view of the main body portion 795, showing the engagement means 805 located at the distal end of the main body portion 795 for holding a shunt device 810 used in the treatment of glaucoma.

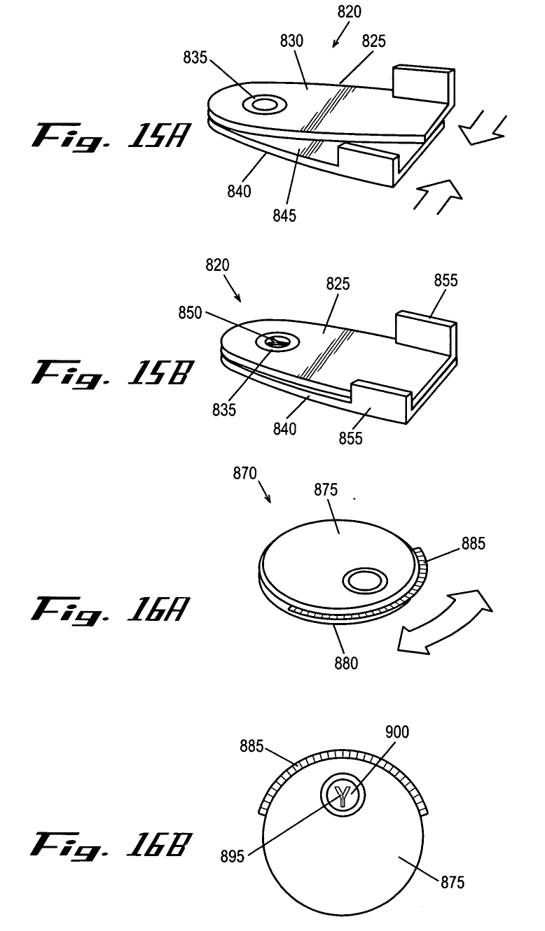

FIG. 15A illustrates an overhead view of a storage apparatus 820. The apparatus comprises a top portion 825 comprising a substantially planar surface 830, having an external key opening 835. The apparatus further comprises a bottom portion 840 comprising a substantially planar surface 845. FIG. 15B illustrates an overhead view of the storage apparatus in the delivery position, showing that when the top portion and bottom portion are rotated, the shunt device 850 used in the treatment of glaucoma is exposed through the external key opening 835 of the top portion. Both the top portion 825 and the bottom portion 840 further comprise a side portion 855 located on opposite sides of the apparatus, which function as an alignment means for the apparatus.

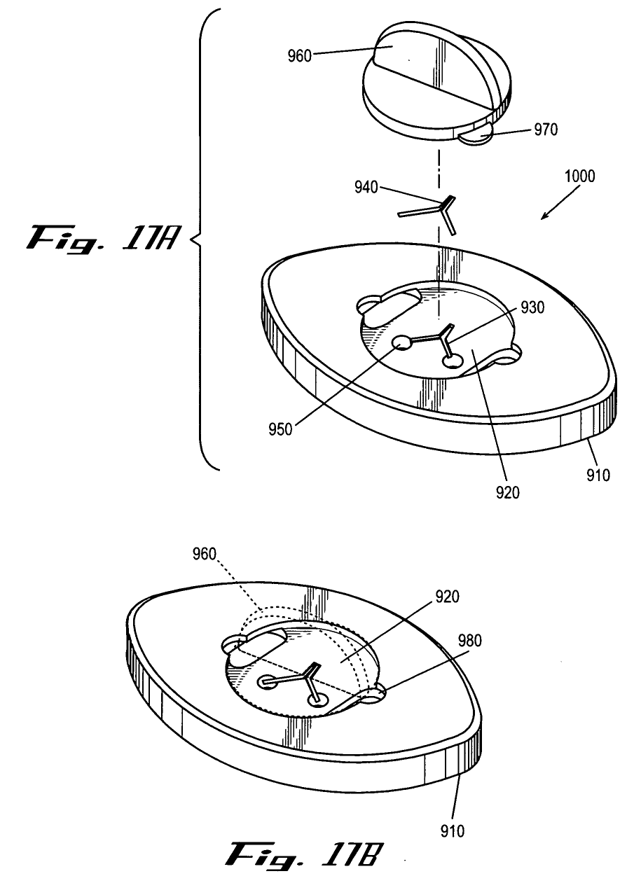

FIG. 16A illustrates an overhead view of a storage apparatus 870. The apparatus comprises a bottom substantially circular planar surface 875; a top parallel substantially circular planar surface 880; and a middle parallel substantially circular planar surface 885. FIG. 16B illustrates an overhead view of the storage apparatus showing that at least part of the middle planar surface 885 extends beyond the top portion and the bottom portion. When the middle planar surface 885 is rotated, the shunt device 895 used in the treatment of glaucoma contained within the middle portion is exposed through the external key opening 900 of the bottom planar surface 875.

FIG. 17A illustrates an overhead view of a storage apparatus 1000. The apparatus comprises a planar base 910 with a recessed portion 920 therein. Within the recessed portion 920 there is at least one further recessed surgical implant device securing means 930. As shown in FIG. 17A, there can be two recessed surgical implant device securing means which are continuous about the shape of the implant stored therein. The securing means 930 is oriented to hold a surgical implant device 940 securely. The recessed portion 920 also has a further recessed portion 950 for aiding in removal of the surgical implant device 940 form its securing means 930. The storage apparatus 1000 possesses a cover 960 for protecting the surgical implant device 940. The cover 960 possesses a shape which allows for mechanical attachment to the base 910. The cover 960 has at least one flange 970 to engage a lip on the base 910. This mechanical engagement allows for ease in removal of the surgical implant device 940 from its protective storage apparatus 1000.

FIG. 17B illustrates the cover 960 and the base 910 engaged so to protect the surgical implant device 940. The lip 980 is positioned so as to releasably engage the flange 970 of the cover 960 of FIG. 17A.

While the above-described embodiments are exemplary, the invention contemplates a wide variety of apparatuses for storage of a surgical implant device. The above-described embodiments are therefore not intended to be limiting to the scope of the equivalents thereof.

What is claimed is:

1. An apparatus for storing a surgical implant device, wherein the apparatus comprises:
a. a base comprising at least one recessed surgical device retaining mount; and
b. a cover configured to releasably engage the base.

2. The apparatus of claim 1, wherein the surgical implant device is a bi-directional shunt for diverting aqueous humor in the eye, and the apparatus comprises two surgical implant retaining mounts for securing the surgical implant device.

3. The apparatus of claim 1, wherein the recessed surgical device retaining mount is continuous over the shape of the surgical implant device.

4. The apparatus of claim 1, wherein the base comprises at least one access area adjacent the recessed surgical device retaining mounts to aid in removal of the surgical implant device.

5. The apparatus of claim 2, wherein the two surgical implant device retaining mounts are recessed into the base at an angle of between about 10° and 150° from one another.

6. The apparatus of claim 2, wherein the two surgical implant device retaining mounts are recessed into the base at an angle of between about 30° and 120° from one another.

7. The apparatus of claim 2, wherein the two surgical implant device retaining mounts are recessed into the base at an angle of between about 45° and 90° from one another.

8. The apparatus of claim 2, wherein the two surgical implant device retaining mounts are between about 0.1 to 2.0 mm in diameter.

9. The apparatus of claim 1, wherein the base contains at least one slot and the cover contains at least one tab for releasably engaging the cover to and from the base.

10. An apparatus for storing a surgical implant device, wherein the apparatus comprises:
a. an elongated barrel having a proximal end with a port, a narrower distal end with a port and a diameter transition portion therebetween;
b. an elongated plunger having a proximal end, a distal end for movable insertion within the narrower distal port of an outer barrel, and a shaft therebetween for insertion within the proximal port of the barrel, wherein the distal end of the plunger comprises at least one recessed surgical implant device retaining mount; and
c. a nosepiece configured to releasably engage the distal portion of the outer barrel.

11. The apparatus of claim 10, further comprising a biasing means to maintain the surgical implant device retaining mounts on the distal end of the plunger protected within the distal end of the barrel when in a relaxed position, and to provide biased resistance to extending the surgical implant device retaining mounts distally beyond the distal port of the outer barrel.

12. The apparatus of claim 11, wherein the biasing means is a coil spring attached at one end to the shaft of the plunger and at another end to the diameter transition portion of the barrel.

13. The apparatus of claim 10 having two surgical implant device retaining mounts, wherein two of the surgical implant device retaining mounts are recessed into the distal end of the plunger at an angle of between about 10° and 150° from one another.

14. The apparatus of claim 10 having two surgical implant device retaining mounts, wherein two of the surgical implant device retaining mounts are recessed into the distal end of the plunger at an angle of between about 30° and 120° from one another.

15. The apparatus of claim 10 having two surgical implant device retaining mounts, wherein two of the surgical implant device retaining mounts are recessed into the distal end of the plunger at an angle of between about 45° and 90° from one another.

16. The apparatus of claim 10 having two surgical implant device retaining mounts, wherein two of the surgical implant device retaining mounts are between about 0.1 and 2.0 mm in diameter.

17. The apparatus of claim 10, wherein the shaft of the plunger is prevented from moving within the narrower distal end of the barrel by abutting the diameter transition portion.

18. The apparatus of claim 10, further comprising a surgical implant device mounted in the distal end of the plunger.

19. The apparatus of claim 18, wherein the surgical implant device is fully enclosed within the barrel and nosepiece when in a storage position, and the surgical implant device is at least partially exposed through the distal port of the barrel by removing the nosepiece and moving the plunger distally when in a delivery position.

20. The apparatus of claim 18, wherein the surgical implant device is a bi-directional shunt for diverting aqueous humor in the eye.
